(19) United States
(12) Patent Application Publication
(10) Pub. No.: US 2009/0093869 A1
(43) Pub. Date: Apr. 9, 2009

(54) MEDICAL DEVICE WITH CURVED STRUTS

(76) Inventors: Brendan Cunniffe, Oranmore (IE); Michael Gilmore, Loughrea (IE); David Slattery, Kinvara (IE); Damian Kelly, Loughrea (IE)

Correspondence Address: RISSMAN JOBSE HENDRICKS & OLIVERIO, LLP, 100 Cambridge Street, Suite 2101, BOSTON, MA, 02114, US

(21) Appl. No.: 11/867,483

(22) Filed: Oct. 4, 2007

(51) Int. Cl. A61F 2/06

(52) U.S. Cl. 623/1.3; 623/1.15

(57) ABSTRACT

A device for implantation in a vessel is provided with a geometry designed to reduce the drag experienced by the device when deployed using a sheathed delivery system. Sections of the device are provided with regions of high-points and low-points on the device's outer surface by curving one or more of the struts of the device. The high points serve as the contact points of the device with the sheath as the sheath and the delivery system are withdrawn from between the device and the vessel.

### RELATED APPLICATIONS

### Field of the Invention

The present invention relates to a medical device having a construction that minimizes drag due to friction when the device is delivered via a sheathed delivery system. More particularly, struts of the medical device are formed to make contact with a ruptured sheath, over a relatively small surface area of the device, as the sheath is withdrawn.

### BACKGROUND OF THE INVENTION

As is known, treatment of vascular blockages due to any one of a number of conditions, such as arteriosclerosis, often involves balloon dilatation and treatment of the inner vessel wall by placement of a stent. These stents are positioned to prevent restenosis of the vessel walls after the dilatation. Other devices, often referred to as drug eluting stents, are now being used to deliver medicine to the vessel wall to also help reduce the occurrence of restenosis.

These stents, i.e., tubular prostheses, typically fall into two general categories of construction. The first category of prosthesis is made from a material that is expandable upon application of a controlled force applied by, for example, a balloon portion of a dilatation catheter upon inflation. The second category of prosthesis is a self-expanding prosthesis formed from, for example, shape memory metals or super-elastic nickel-titanium (NiTi or Nitinol) alloys, that will automatically expand from a compressed or restrained state when the prosthesis is advanced out of a delivery catheter and into the blood vessel.

Some known prosthesis delivery systems for implanting self-expanding stents include an inner lumen upon which the compressed or collapsed prosthesis is mounted and an outer restraining sheath that is initially placed over the compressed prosthesis prior to deployment. When the prosthesis is to be deployed in the body vessel, the outer sheath is moved in relation to the inner lumen to “uncover” the compressed prosthesis, allowing the prosthesis to move to its expanded condition. Some delivery systems utilize a “push-pull” type technique in which the outer sheath is retracted while the inner lumen is pushed forward. Still other systems use an actuating wire that is attached to the outer sheath.

Delivery systems are known where a self-expanding stent is kept in its compressed state by a sheath positioned about the prosthesis. A balloon portion of the delivery catheter is provided to rupture the sheath and, therefore, release the prosthesis. For example, in U.S. Pat. No. 6,656,213, the stent may be provided around the balloon, with the sheath around the stent, that is, the balloon, stent, and sheath are co-axially positioned, such that expansion of the balloon helps to expand the self-expanding stent as well as rupture the sheath.

Once the balloon is inflated and the sheath is ruptured, the stent expands to its non-compressed state. The ruptured sheath, however, is now positioned between the expanded stent and the vessel wall. In some systems, the sheath is left in place, either permanently or to bio-degrade over time. In other systems, the sheath is attached to the delivery catheter and is withdrawn when the delivery catheter is withdrawn from the vessel.

When withdrawing the ruptured sheath, the sheath may contact the deployed stent on its outside surface. As a result, the frictional force between the stent and the sheath results in a retraction force on the stent upon withdrawal of the catheter. This force can serve to reduce the ability of the stent to remain anchored at the target site.

There is, therefore, a need for a mechanism to minimize the effects of the withdrawal of a ruptured sheath from between a vessel wall and an expanded stent without affecting the delivered stent's functionality.

### SUMMARY OF THE INVENTION

The present invention serves to address the problem presented by the movement of the sheath with respect to the stent by providing the stent with a geometry designed in such a way so as to provide for regions of high-points and low-points on the stent outer surface. The high points serve as the contact points with the retaining sheath as it is withdrawn from the vessel.

In one embodiment, a device for implantation in a vessel comprises a series of struts configured in a circumferential pattern comprising peaks and valleys defining a lumen of the device, each strut having an outer surface and an inner surface, the inner surfaces generally defining an inner outline of the device, where at least one strut is curved along a radial direction with respect to the lumen.

The at least one strut is curved so as to extend below the inner outline and into the lumen. The outer surfaces generally define an outer outline of the device and at least one strut comprises a curved portion extending beyond the outer outline and away from the lumen.

In another embodiment, a first circumferential band of struts in which the at least one curved strut is located; and a second circumferential band of struts in which no strut is curved are provided.

In another embodiment, a first longitudinal stripe of struts in which the at least one curved strut is located; and a second longitudinal stripe of struts in which no strut is curved are provided.

In another embodiment, a first circumferential band of struts in which the at least one curved strut is located and a first longitudinal stripe of struts in which the at least one curved strut is located are provided where struts not located within either the first circumferential band of struts or the first longitudinal stripe of struts are not curved.

In one embodiment, a device for implantation in a vessel includes: a series of struts configured in a circumferential pattern comprising peaks and valleys and defining a lumen of the device, each strut having an outer surface and an inner surface, the outer surfaces generally defining an outer outline of the device, where at least one strut comprises a first high point that extends beyond the outer outline and away from the lumen.

The inner surfaces generally define an inner outline of the device and at least one strut comprises a portion extending below the inner outline and into the lumen.

In one embodiment, the at least one strut comprising the first high point further comprises: a second high point that extends beyond the outer outline of the device.

In yet another embodiment, a device for implantation in a vessel comprises: a radially expandable portion formed of a plurality of struts arranged in a circumferential pattern; and a lumen extending longitudinally through the device and defined by the radially expandable portion, where at least one strut in the radially expandable portion comprises: a first portion and a second portion, each of the first and second portions curved in a direction away from the lumen.

The radially expandable portion may comprise a shape-memory material.

In another embodiment, a method of forming an implantable device comprising at least one strut having reduced drag characteristics with respect to a delivery sheath comprises: providing a first texture pattern and a second texture pattern with each of the first and second texture patterns configured to complement the other. The device is positioned between the first and second texture patterns and a shape corresponding to the first and second texture patterns is imparted to at least one strut of the device.

In one embodiment, the method further comprises: providing the first and second texture patterns on female and male portions of a forming tool, respectively, to provide a curve to at least one strut of the device.

### BRIEF DESCRIPTION OF THE DRAWINGS

The above and further advantages of the present invention may be better understood by referring to the following description in conjunction with the accompanying drawings in which:

FIG. 1 is a representation of a known ostial protection devices;

FIG. 2 is a representation of a known device delivery system;

FIG. 3 is a cross-section view of the delivery system of FIG. 2;

FIGS. 4 and 5 represent operation of the delivery system of FIG. 2 in a vessel;

FIG. 6 is a cross-section view of the delivery system as shown in FIG. 5;

FIG. 7 is a magnified representation of interaction of struts of a known device with a sheath in a vessel;

FIG. 8A is a magnified representation of interaction of struts according to one embodiment of the present invention with a sheath in a vessel;

FIG. 8B is a magnified representation of interaction of struts according to another embodiment of the present invention with a sheath in a vessel;

FIG. 9 is an isometric representation of a strut portion of the device shown in FIG. 1;

FIG. 10 is an isometric representation of a strut portion of the device according to one embodiment of the present invention;

FIGS. 11 and 12 are representations of a forming tool;

FIG. 13 is a representation of a forming tool according to an embodiment of the present invention;

FIG. 14 is a representation of a forming tool according to another embodiment of the present invention;

FIG. 15 is a representation of a device including curved or rippled struts in accordance with one embodiment of the present invention;

FIG. 16 is a cross-section view of the device shown in FIG. 15; and

FIGS. 17A-17C represent alternate embodiments of the present invention.

### DETAILED DESCRIPTION

The motion of the ruptured sheath and the catheter upon being withdrawn can interfere with the proper placement of the delivered medical device, e.g., a stent. The present invention serves to address the problem presented by providing a stent with a form or shape that minimizes surface contact between the sheath and the stent.

The geometry of the stent in accordance with one embodiment of the present invention, is designed in such a way so as to provide for regions of high-points and low-points on the stent outer surface. The high points serve as the contact points with the retaining sheath. In addition, the low-points on the surface of the stent provide a location where a drug/polymer coating can be located. Further, the high points may assist with the anchorage of the device on the vessel wall as each high point may function similar to a “mini-stud” on the outside surface of the stent. This additional anchorage may help to reduce any tendency for the device to move out of position once deployed at the target location in the vessel.

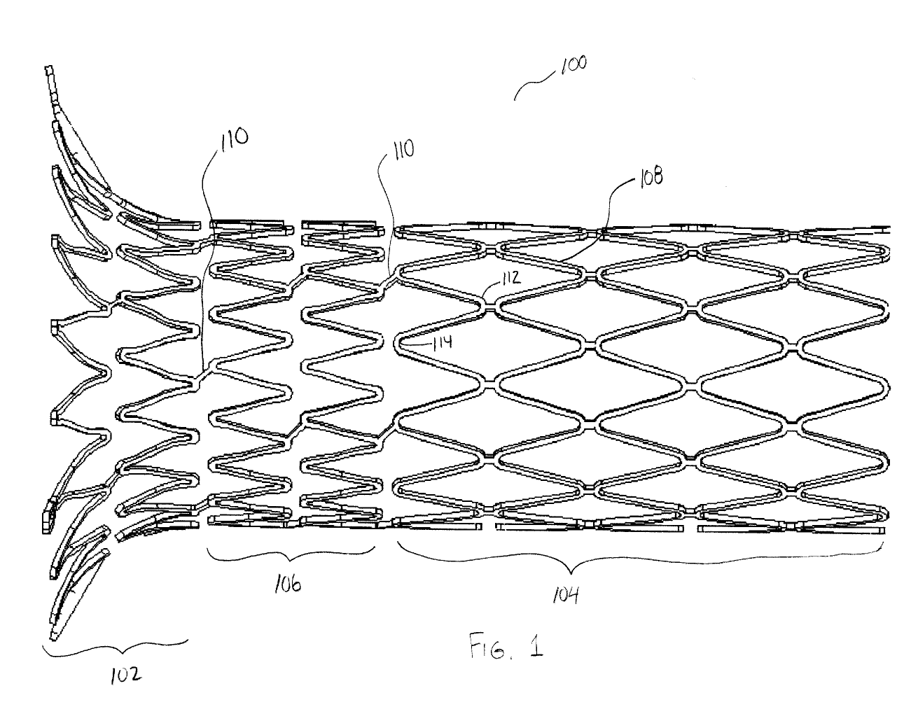

Reference is now made to FIG. 1, which illustrates a schematic view of an intraluminal device 100, for example, an ostial protection device as described in US Publication 20070061003A1 published Mar. 15, 2007, for “Segmented Ostial Protection Device,” and which is herein incorporated by reference in its entirety. It should be noted that while the present description is with reference to an ostial protection device, the claims are not limited to only medical devices intended for insertion at an ostium. The intraluminal device 100 includes a cap or flared portion 102, an anchor portion 104, and an articulating portion 106. The anchor portion 104 is configured to fit into a side-branch vessel and the cap portion 102 is configured to selectively protect at least part of an ostial region. The articulating portion 106 flexibly connects the anchor portion 104 to the cap portion 102, such that various angles of articulation are possible between each of the three portions. The articulating portion 106 includes connectors 110 connecting to the cap portion 102 and to the anchor portion 104.

The intraluminal device 100 may be configured to protect an ostial region and/or a side branch vessel by selectively covering at least part of an inner wall of the ostial region. This positioning may prevent a plaque layer or parts thereof from migrating into the side branch vessel by the “snow-plow” effect, which may result from applying an angioplasty device.

The intraluminal device 100 may be formed of a generally elastic, super-elastic, in-vivo stable and/or “shape-memorizing” material, i.e., a material able to be initially formed in a desired shape, e.g., during an initial procedure performed at relatively high temperature, to be deformed, e.g., compressed, and to assume the desired shape in which it was previously shaped. The intraluminal device 100 may be formed of Nickel-Titanium alloy (“Nitinol”) that possesses both super-elastic and shape-memorizing properties. Biocompatible non-elastic materials, such as stainless steel, for example, may be also used. Other combinations of materials and processes would be understood by one of ordinary skill in the art.

The intraluminal device 100 may be formed from a wire or cut from a single tube of material. The intraluminal device 100 may be formed from a single piece of material or may be assembled in sections. In general, each section comprises a plurality of struts 108 arranged in a manner of peaks 112 and valleys 114 familiar to those of ordinary skill in the art and as described in the above-incorporated '003 publication.

The struts 108 may have a cross-section that is, but not limited to, circular, oval, rectangular, or square. One of ordinary skill in the art will understand the options available with respect to the cross-section chosen for the struts 108 depending upon the intended application of the device.

The self-expanding device 100 may be delivered via a system that uses a sheath and a balloon portion of a delivery catheter. In general, and which will be explained in more detail below, the device 100 is compressed and loaded in a low-profile or crimped state about a balloon portion and surrounded by a sheath. The balloon portion is inflated, causing the sheath to rupture and release the constrained device 100 into its expanded condition.

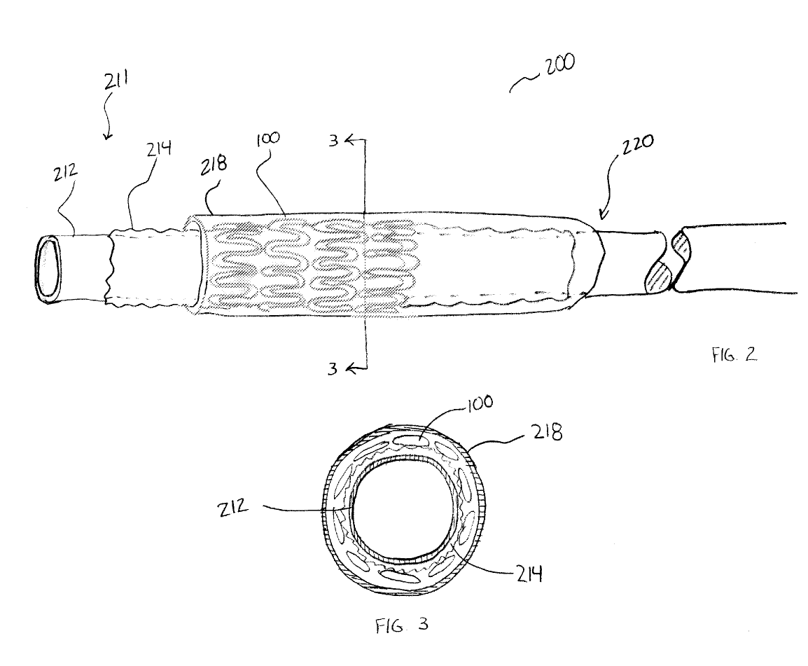

A medical device delivery system 200, as shown in FIG. 2, includes a delivery catheter 212 with a balloon portion 214 positioned at a distal end 211 of the catheter 212. As is known, a lumen is provided to inflate the balloon 214 as necessary during the procedure to deliver the device 100 that is placed at the distal end of the catheter 212 and around the balloon 214. As per the present discussion, the device 100 is a self expanding device and, therefore, a cylindrical sheath 218 is also disposed at the distal end 211 of the catheter 212 so as to enclose the device 100 and the balloon 214. The sheath 218 is attached to the catheter 212 at an attach location 220 proximal to the distal end 211 of the catheter 212. In one embodiment of the present invention, the attach location 220 is proximal to the balloon portion 214.

A cross-section view of the system 200, along line 3-3, is presented in FIG. 3. As shown, the sheath 218 surrounds the stent or device 100 and the balloon 214 positioned on the catheter 212.

The sheath 218 may be made from a material having a grain, or fibers, that can be longitudinally oriented, for example, PTFE. Other materials may be used for the sheath as understood by one of ordinary skill in the art.

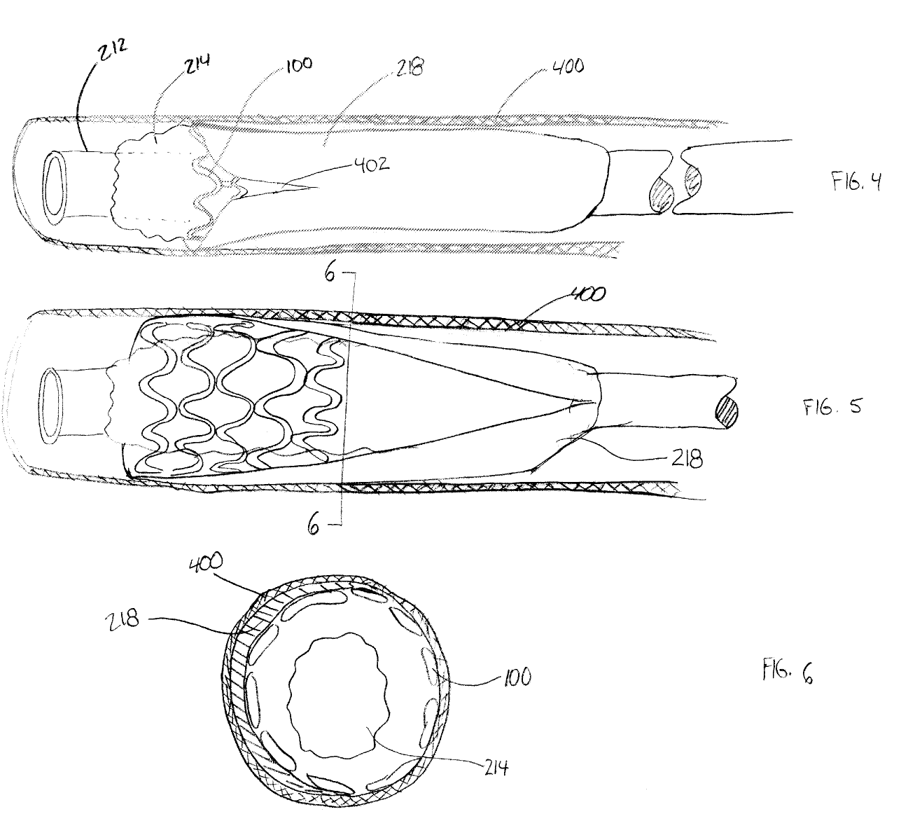

Referring now to FIG. 4, the delivery system 200 is positioned at a desired location within a vessel 400. The balloon portion 214 is inflated causing the sheath 218 to rupture. As the sheath 218 ruptures, the device 100 is released to expand within the vessel 400. The sheath 218 will rupture or split, as shown in FIG. 5, and due to the elastic properties of the sheath 218, will no longer constrain the device 100. In general, the sheath 218, upon expansion of the balloon 214, will tear or rupture along a perforation or initial cut 402 in substantially a straight line following a longitudinal axis of the sheath 218 as defined, generally, by the catheter 212.

The sheath 218 is made from a plastic material and, as above, is generally cylindrical. Once the sheath 218 ruptures, however, it is no longer a cylinder and has a form that covers less than all of the circumference of the now-expanded stent 100. Referring to FIG. 6, a cross-section view of the system 200 of FIG. 5 along the line 6-6, the now-deflated balloon portion 214 is within the lumen of the expanded stent 100. The ruptured sheath 218 is trapped between a portion of the now-expanded stent 100 and the vessel wall 400. The ruptured sheath 218, however, is only trapped between the stent 100 and the vessel wall 400, for a portion, i.e., less than all, of the circumference of the now-expanded stent 100. When the catheter, and the now-deflated balloon portion 214, are withdrawn, that portion of the ruptured sheath 218 trapped between the stent 100 and the vessel wall 400 may pull on the deployed stent 100 and interfere with its proper placement.

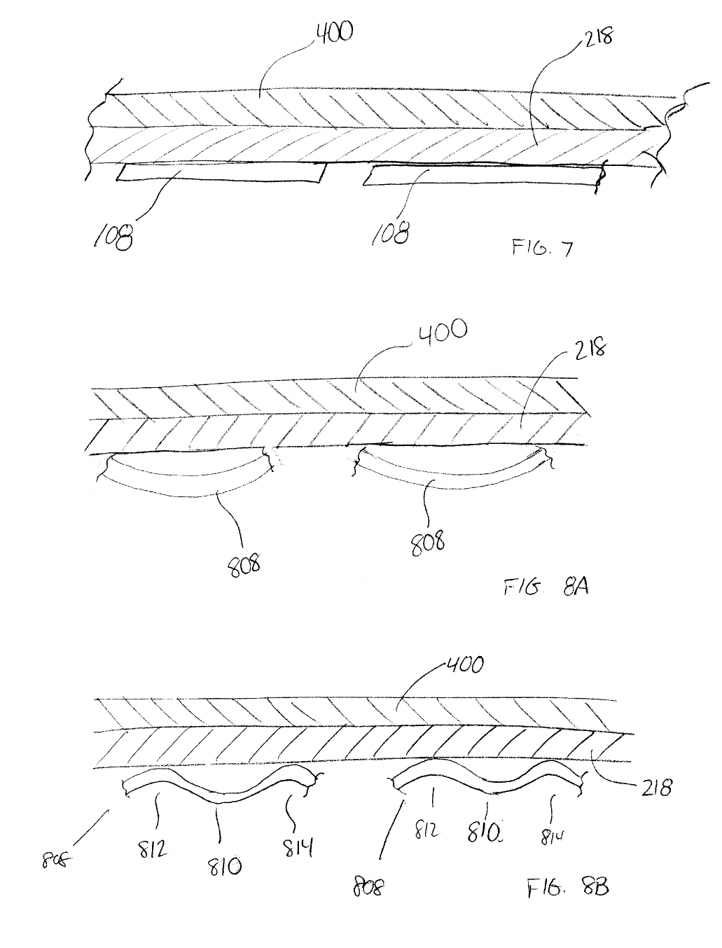

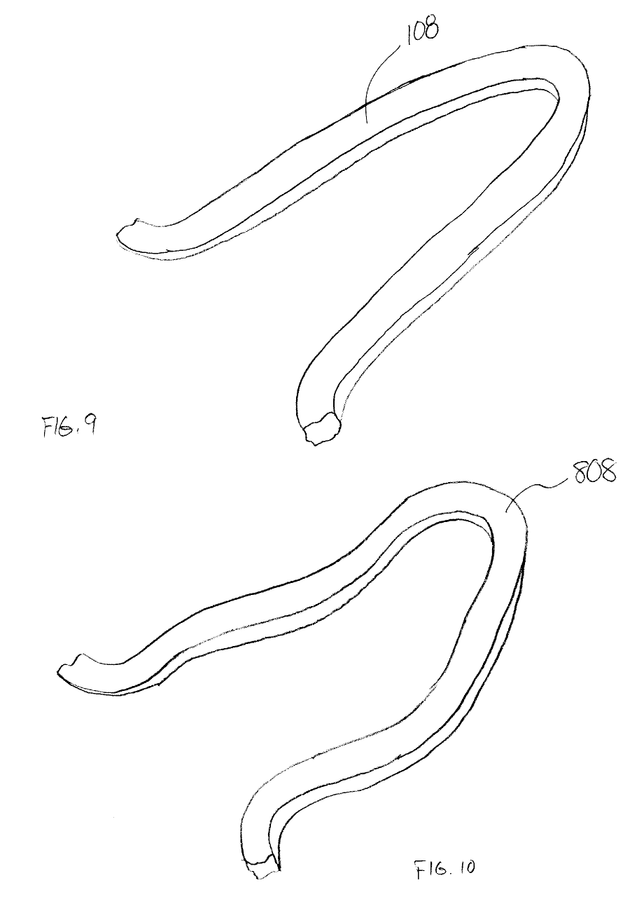

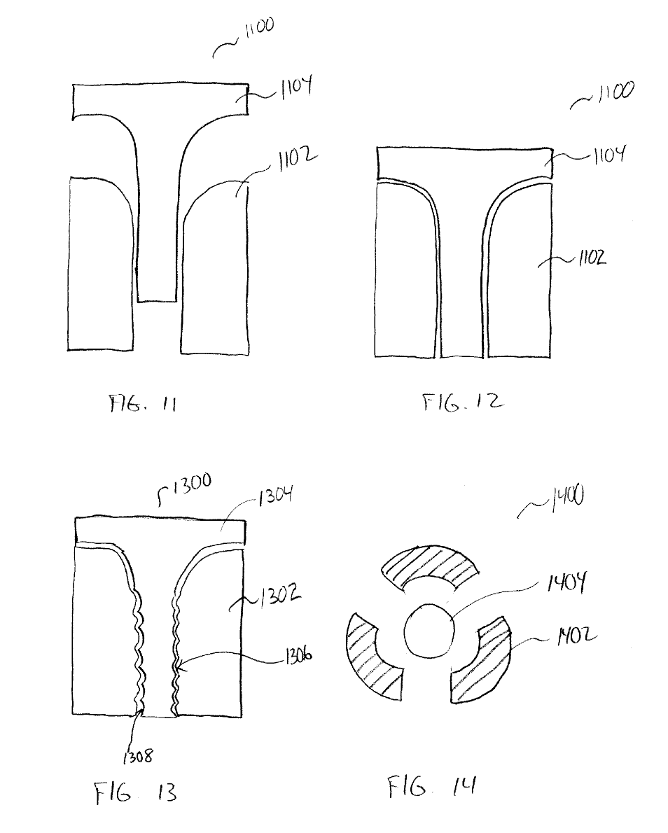

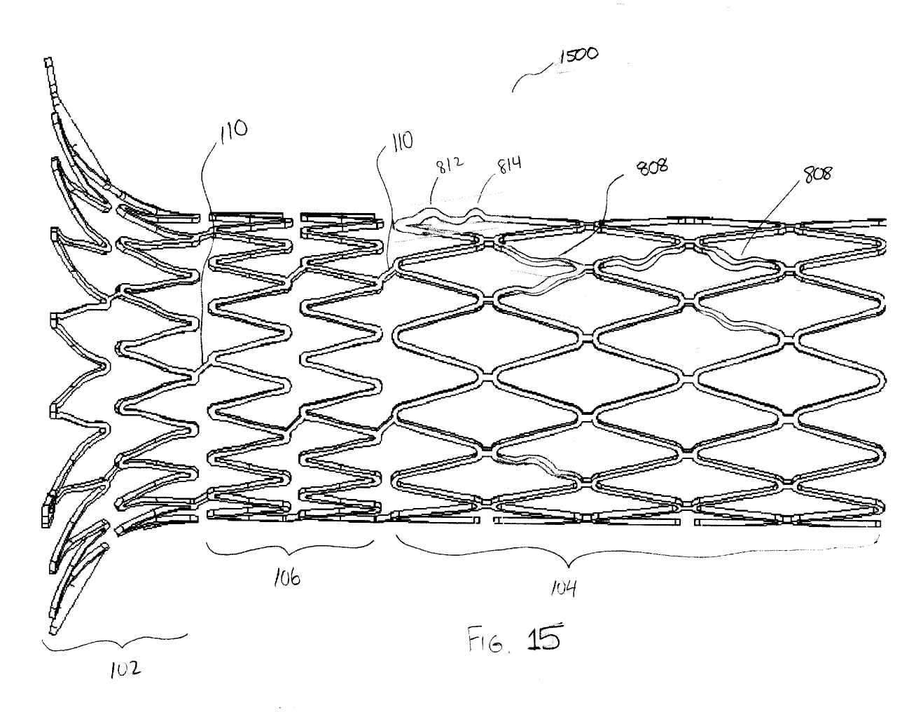

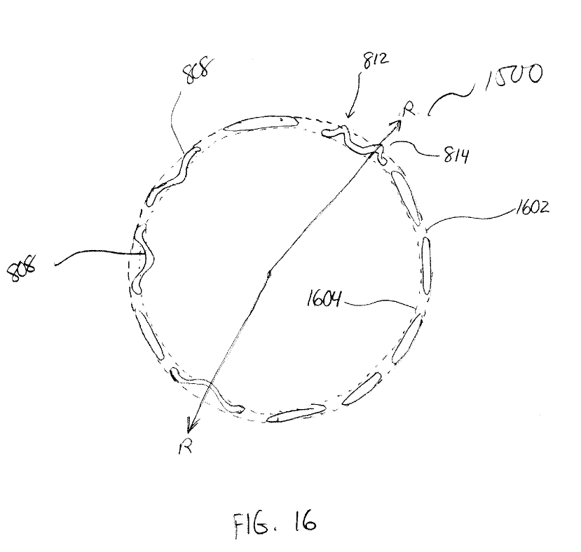

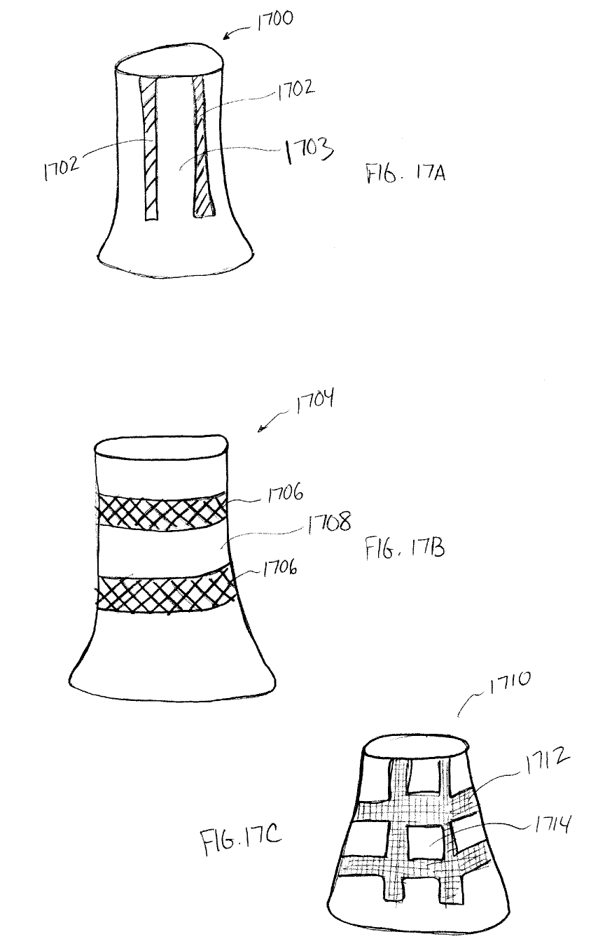

A magnified view of the relationship between the struts 108 of the device 100 and the ruptured sheath 218 caught between the struts 108 and the vessel 400 is presented in FIG. 7. The struts 108 have a generally planar structure that presents a relatively large surface area to the ruptured sheath 218 material. Of course, a total amount of friction between the struts 108 and the sheath 218 depends upon the number of struts in the device 100 and each strut's surface area.

There is intimate contact between the stent and the retaining sheath (and any crimping/processing tools that must be used in the processing of the device) with this known method of stent delivery. As a result, it is possible that any drug/polymer coating deposited on the outer surface of the stent will be removed during routine processing of the device and during withdrawal of the sheath from the body post-deployment.

In one embodiment of the present invention, the struts are provided with a portion having a radius of curvature or “ripple” that reduces or minimizes an amount of surface area that is in contact with the sheath 218. Referring now to FIG. 8A, a medical device such as device 100 described above, is provided with struts 808 that are curved or rippled so as to not present a large surface area to the sheath 218. This ripple has areas of high points and low points. The high points contact the sheath while the low points are away from, i.e., not in contact with, the sheath. The low points of the outer surface, therefore, are protected from damage and it is less likely that any coating applied to these regions will be damaged through contact with the sheath. The curve applied to the strut resulting in the low point in which a coating may be applied is different from those known devices having through-holes, divots or reservoirs in which coating is placed. In those known devices, a portion of the strut material is removed and the strut remains substantially planar. In contrast, in some embodiments of the present invention, the strut is not planar due to the curved portion or portions.

Referring now to FIG. 8B, in another embodiment, the strut 808 includes a first portion 810 that is curved in a direction away from the outer surface of the strut, i.e., away from the sheath 218. In some embodiments, the first portion 810 may extend into the lumen of the device. Second and third portions, 812, 814, respectively, curve away from the lumen, i.e., toward the sheath 218, to provide high points on the outer surface. As above, this “wave” construction provides for smaller areas of contact between the stent and the sheath 218. The high points are shown as being in contact with the sheath 218, thereby presenting a lower amount of a surface area and, similar to the above, any coating applied in the first portion 810 is less likely to be disrupted by relative motion of the sheath. It should be noted that the representations of the struts 808 as shown in FIGS. 8A and 8B are for explanatory purposes only and not intended to be limiting or to represent a scale drawing.

As above, a strut 108, as shown in FIG. 9, presents a substantially planar profile to the sheath 218. In contrast, referring now to FIG. 10, the strut 808, due to its curvature, presents a lower amount of mechanical contact, i.e., frictional contact, due to reduced surface areas of contact, to the sheath 218.

As shown in FIG. 15, a device 1500, similar to the device 100 of FIG. 1, includes a number of struts 808 that are each curved so as to minimize contact with the sheath 218 upon its withdrawal. It should be noted that not all of the struts shown on the device 1500 include curves. In one embodiment of the present invention, less than all of the struts are curved. Further, curved struts 808 may be placed only in a specific section of the device 1500, for example, the anchor portion 104. The curved struts 808 may be placed within a region in any predetermined pattern. In one embodiment, all of the struts in a device may be provided as curved struts 808.

Alternately, as shown in FIG. 17A, in one embodiment, a device 1700 may have one or more longitudinal striped areas 1702 where the struts (not shown) within those stripes 1702 are curved. Further, other longitudinal stripe areas 1703 are provided where no struts are curved. The provision of longitudinal stripes 1702 may provide for easier withdrawal of the deflated sheath. Still further, in another embodiment, as depicted in FIG. 17B, a device 1704 may have one or more circumferential bands 1706 where the struts (not shown) within those bands 1706 are curved. The provisional of the circumferential bands 1706 in which the struts are curved may be beneficial to the provision of medicinal coatings applied to the device. Similar to the longitudinal striped areas, one or more bands 1708 may define areas in which no struts are curved. In yet another embodiment, shown in FIG. 17C, a device 1710 includes circumferential bands and longitudinal stripes resulting in a cross-hatch or checkerboard pattern 1712 where struts with curves are located and other areas 1714 where struts are located but are not curved.

A cross-section view of the device 1500 is presented in FIG. 16. As represented by a dotted-line 1602, the device has a general outer outline and, as represented by a dotted-line 1604, a general inner outline. The inner and outer outlines are, conceptually, defined by the struts that are not curved in accordance with embodiments of the present invention. In this representation, looking through the lumen of the device 1500, the curved struts 808 may extend, i.e., be an intrusion, into the lumen or, as shown, extend inwardly past the general inner outline 1604. This intrusion, however, is not significant enough to interfere with flow of fluid through the stent 1500 once implanted in a vessel. Further, the high points, i.e., the second and third portions 812, 814, extend, or protrude, “beyond” the general outer outline 1604. The amount of protrusion, however, is not sufficient to cause injury to the vessel wall in which the device is placed. The “curve,” either in the direction toward or away from the lumen can be considered as along a radius or diameter across the lumen as represented by the arrow R in FIG. 16. The representation as shown in FIG. 16, it should be noted, is not to scale and aspects of the figures are emphasized to aid in the explanation of embodiments of the present invention.

It should be noted that the high points may be linearly arranged along some portion, or all, of the longitudinal length of the device and over which the ruptured sheath could then “ride along” as it is withdrawn. The high points also could be placed in a predetermined pattern and might be provided only on one side of the device. As one non-limiting example, the high points might be provided on one circumferential half of the device and then oriented opposite the initiation slit in the sheath. It would then be expected that the ruptured sheath would then be trapped between the device and the vessel wall opposite the slit and where the high points are positioned.

A forming tool 1100, shown in FIGS. 11 and 12, is used to form the device 100 of FIG. 1. The forming tool 1100 includes a female form portion 1102 and a male form portion 1104. The device 100, being manufactured from Nitinol, is shape-set using the forming tool 1100. A laser-cut tubular device (not shown) is positioned between the female form portion 1102 and the male form portion 1104 while being exposed to a high temperature (typically≈500° C.) over a certain time period (typically approximately 10 minutes). As a result, the cylindrical shape of the laser-cut tubular profile is turned into the shape of the device 100. One of ordinary skill in the art will understand the forming of Nitinol-based devices.

To create the curved or rippled struts of the present invention, a textured forming tool 1300, as shown in FIG. 13, is provided. Similar to the forming tool 1100, the textured forming tool 1300 includes a textured female portion 1302 and a textured male forming portion 1304 having complementary textured profiles 1306, 1308, respectively. The textured/rippled profiles 1306, 1308 introduce the curve or ripple to the stent struts. Of course, if either stripes 1702 or bands 1706 of curved struts, or any other pattern, are desired, the female and/or male portions 1302, 1304 would be designed accordingly. The time-temperature shape setting process, similar to that described above, and known to those of ordinary skill in the art, will be sufficient to have these curves or ripples remain in the device 1500.

The provision of curved or rippled struts, however, requires a more complex forming tool arrangement then the two-part tool shown in FIGS. 11 and 12. With reference to the device 100, the straight nature of the struts of the anchor on the device allows the male forming portion 1104 to easily slide inside the female forming portion 1102. The introduction of the curve or ripple strut 808 will not allow such easy motion. In one embodiment, a forming tool 1400, as shown in FIG. 14, includes a female forming portion 1402 split into multiple parts, for example, three symmetrical parts. A male forming portion 1404 is similar to the previously described male forming portions. The parts of the female forming portion 1402 are then provided around the male part once the blank form has been mounted on the male part 1404.

It is to be understood that the present invention is not limited in its application to the details of construction and the arrangement of the components set forth in the foregoing description or illustrated in the drawings. The invention is capable of other embodiments or of being practiced or carried out in various ways. Specifically, while the foregoing description was with respect to a flared ostial protection device, the features described here can equally be applied to other types of devices, e.g., a straight cylindrical main-branch stent. Further, some struts may be curved differently from other curved struts on the same device.

Also, it is to be understood that the phraseology and terminology employed herein are for the purpose of description and should not be regarded as limiting.

It is further appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although various exemplary embodiments of the present invention have been disclosed, it will be apparent to those skilled in the art that changes and modifications can be made that will achieve some of the advantages of the invention without departing from the spirit and scope of the invention. It will be apparent to those reasonably skilled in the art that other components performing the same functions may be suitably substituted.

What is claimed is:

1. A device for implantation in a vessel, the device comprising:
a series of struts configured in a circumferential pattern comprising peaks and valleys and defining a lumen of the device, each strut having an outer surface and an inner surface, the inner surfaces generally defining an inner outline of the device,
wherein at least one strut is curved along a radial direction with respect to the lumen.

2. The device of claim 1, wherein the at least one strut is curved so as to extend below the inner outline and into the lumen.

3. The device of claim 1, wherein the outer surfaces generally define an outer outline of the device and at least one strut comprises a curved portion extending beyond the outer outline and away from the lumen.

4. The device of claim 1, further comprising:
a first circumferential band of struts in which the at least one curved strut is located; and
a second circumferential band of struts in which no strut is curved.

5. The device of claim 1, further comprising:
a first longitudinal stripe of struts in which the at least one curved strut is located; and
a second longitudinal stripe of struts in which no strut is curved.

6. A device for implantation in a vessel, the device comprising:
a series of struts configured in a circumferential pattern comprising peaks and valleys and defining a lumen of the device, each strut having an outer surface and an inner surface, the outer surfaces generally defining an outer outline of the device,
wherein at least one strut comprises a first high point that extends beyond the outer outline and away from the lumen.

7. The device of claim 6, wherein the inner surfaces generally define an inner outline of the device and at least one strut comprises a portion extending below the inner outline and into the lumen.

8. The device of claim 6, wherein the at least one strut comprising the first high point further comprises:
a second high point that extends beyond the outer outline of the device.

9. The device of claim 6, further comprising:
a first longitudinal stripe of struts in which at least one strut comprises a respective first high point; and
a second longitudinal stripe of struts in which no strut comprises a respective first high point.

10. A device for implantation in a vessel, the device comprising:
a radially expandable portion formed of a plurality of struts arranged in a circumferential pattern; and
a lumen extending longitudinally through the device and defined by the radially expandable portion,
wherein at least one strut in the radially expandable portion comprises:
a first portion and a second portion, each of the first and second portions curved in a direction away from the lumen.

11. The device of claim 10, wherein the at least one strut with the first and second portions further comprises:
a third portion positioned along the at least one strut and between the first and second portions,
wherein the third portion is curved in a direction toward the lumen.

12. The device of claim 11, wherein:
the third portion of the strut extends into the lumen.

13. The device of claim 10, wherein each strut comprises an outer surface and an inner surface, the outer surfaces generally defining an outer outline of the device and wherein the first and second portions extend beyond the outer outline and away from the lumen.

14. A method of forming an implantable device comprising at least one strut having reduced drag characteristics with respect to a delivery sheath, the method comprising:
providing a first texture pattern and a second texture pattern provided thereon, each of the first and second texture patterns configured to complement the other;
positioning the implantable device between the first and second texture patterns; and
imparting a shape corresponding to the first and second texture patterns to at least one strut of the device.

15. The method of claim 14, further comprising:
providing a forming tool having a male portion and a female portion;
providing the female portion with the first texture pattern;
providing the male portion with the second texture pattern; and
positioning the implantable device between the male portion and the female portion to impart the shape corresponding to the first and second texture patterns to at least one strut of the device.

16. The method of claim 15, further comprising:
providing the first and second texture patterns on the female and male portions, respectively, to provide a curve to at least one strut of the device for implantation.

17. The method of claim 15, wherein the device comprises a shape-memory material and imparting the shape comprises:
exposing the device to an elevated temperature for a period of time.

18. A device for implantation in a vessel, the device comprising:
a radially expandable portion formed of a plurality of struts arranged in a circumferential pattern; and
a lumen extending longitudinally through the device and defined by the radially expandable portion,
wherein at least one strut in the radially expandable portion comprises:
a first curved portion, the first curved portion curved in a direction away from the lumen.

19. The device of claim 18, wherein the at least one strut with the first curved portion further comprises a second curved portion also curved in a direction away from the lumen.

20. The device of claim 19, wherein the at least one strut with the first and second curved portions further comprises:
a third curved portion positioned along the at least one strut and between the first and second curved portions.

21. The device of claim 20, wherein the third curved portion is curved in a direction toward the lumen.

22. The device of claim 21, wherein:
the third curved portion of the strut extends into the lumen.

23. The device of claim 18, wherein each strut comprises an outer surface and an inner surface, the outer surfaces generally defining an outer outline of the device and wherein the first and second curved portions extend beyond the outer outline and away from the lumen.

24. The device of claim 18, further comprising:
a plurality of struts comprising respective first curved portions,
wherein the plurality of struts are arranged within a longitudinal stripe along a portion of a length of the device.

25. The device of claim 18, further comprising:
a first circumferential band of struts in which the at least one curved strut is located; and
a first longitudinal stripe of struts in which the at least one curved strut is located,
wherein struts not located within either the first circumferential band of struts or the first longitudinal stripe of struts are not curved.
